(19) United States
(12) Patent Application Publication
(10) Pub. No.: US 2014/0301989 A1
(43) Pub. Date: Oct. 9, 2014

(54) METHODS FOR INCREASING THE POTENCY AND EFFICACY OF STEM CELLS

(71) Applicants: Brian Johnstone, Indianapolis, IN (US); March Keith, Carmel, IN (US)

(72) Inventors: Brian Johnstone, Indianapolis, IN (US); March Keith, Carmel, IN (US)

(21) Appl. No.: 14/127,452

(22) PCT Filed: Jun. 18, 2012
(86) PCT No.: PCT/US12/42952

§ 371 Date: Dec. 20, 2013

Related U.S. Application Data
(60) Provisional application No. 61/498,079, filed on Jun. 17, 2011.

(51) Int. Cl. C12N 5/0775; C12N 15/85; A61K 35/12; C12N 15/86

(52) U.S. Cl. CPC C12N 5/0667; C12N 15/86; C12N 15/85; A61K 35/35
USPC 424/93.21; 424/93.7; 435/289.1

(57) ABSTRACT

The disclosure of the present application provides methods and kits for the increase in efficacy of adipose stromal cells. In at least one embodiment, the present disclosure includes disclosure of a method of cell-based therapy, the method comprising the steps of exposing at least one mammalian stem cell expressing mesenchymal-epithelial transition factor (c-Met) to a compound operable to phosphorylate c-Met and administering the at least one mammalian stem cell to a patient.

### PRIORITY

The present International Patent Application is related to, and claims the priority benefit of, U.S. Provisional Patent Application Ser. No. 61/498,079, filed on Jun. 17, 2011, the contents of which are hereby incorporated into the present disclosure in their entirety.

### STATEMENT REGARDING FEDERALLY SPONSORED RESEARCH AND DEVELOPMENT

This invention was made with government support from the National Institutes of Health under grant number HL 077688. The U.S. Government has certain rights in the present disclosure.

### BACKGROUND

The discovery of pluripotent cells in adipose tissue has revealed a novel source of cells that may be used for autologous cell therapy to regenerate tissue. These pluripotent cells reside in the “stromal” or “non-adipocyte” fraction of the adipose tissue; they were previously considered to be pre-adipocytes, i.e. adipocyte progenitor cells, however recent data suggest a much wider differentiation potential. Zuk et al. were able to establish differentiation of such subcutaneous human adipose stromal cells (“ASCs”) in vitro into adipocytes, chondrocytes and myocytes. Zuk P A, et al. Mol Biol Cell 13(12):4279-4295, 2002. These findings were extended in a study by Erickson et al., which showed that human ASCs could differentiate in vivo into chondrocytes following transplantation into immune-deficient mice. Erickson G R, el al. Biochem Biophys Res Commun. 2002; 290:763-669. More recently, it was demonstrated that human ASCs were able to differentiate into neuronal cells, osteoblasts cardiomyocyte, and endothelial cells. The key benefit of using pluripotent ASCs for autologous cell therapy is the ease by which they can be isolated and their relative abundance.

### SUMMARY

In at least one embodiment of a method of cell-based therapy of the present disclosure, the method comprises the steps of exposing at least one mammalian stem cell expressing mesenchymal-epithelial transition factor (c-Met) to a compound operable to phosphorylate c-Met, and administering the at least one mammalian stem cell to a patient, where the patient may have a vascular deficiency. In at least one embodiment, the step of administering the at least one mammalian stem cell treats the patient's vascular deficiency. Further, the at least one mammalian stem cell may originally be isolated from the patient. Optionally, the at least one mammalian stem cell may be isolated from a source selected from the group consisting of adipose tissue, peripheral blood, blood vessels, and umbilical cord blood. The at least one mammalian stem cell in an exemplary embodiment may comprise an adipose stromal cell. Additionally, in at least one embodiment of the method of cell-based therapy of the present disclosure, the compound may be at least one of hepatocyte growth factor (HGF), a fragment of HGF operable to bind to c-Met, a monoclonal antibody specific for c-Met, a c-Met agonist (such as a small molecule or nucleic acid), or a stressing agent, such as one comprising hydrogen peroxide.

In at least one embodiment of a method of cell-based therapy of the present disclosure, the step of exposing the at least one mammalian cell may be for at least one minute, for at least fifteen minutes, or for at least about one hour. Additionally, the step of administering the at least one mammalian stem cell of the present disclosure may comprise intravenously injecting the at least one mammalian stem cell into the patient, where the patient may be a mammal, such as a human. Further, the step of administering may, in at least one embodiment of the present disclosure, cause at least one of a regeneration of at least one tissue of the patient, and/or a relative increase in blood perfusion of an ischemic tissue in the patient.

In at least one embodiment of a method of cell-based therapy of the present disclosure, the method comprises the steps of exposing at least one mammalian stem cell expressing mesenchymal-epithelial transition factor (c-Met) to an environmental condition operable to phosphorylate c-Met, and administering the at least one mammalian stem cell to a patient, wherein the environmental condition may in at least one embodiment be is selected from a group consisting of hypoxia, and serum starvation.

In at least one embodiment of a method of treating a patient of the present disclosure, the method comprises the steps of exposing at least one adipose stromal cell to an effective amount of an agonist for mesenchymal-epithelial transition factor (c-Met), and administering the at least one adipose stromal cell to treat a patient in need. The at least one mammalian stem cell of the present disclosure may be isolated from a source selected from the group consisting of adipose tissue, peripheral blood, blood vessels, and umbilical cord blood. Additionally, the at least one mammalian stem cell may be isolated from the patient. Further, the at least one adipose stromal cell may be capable of regenerating at least one tissue of the patient. Moreover, the step of administering the at least one mammalian cell may cause a relative increase in blood perfusion of an ischemic tissue in the patient.

In at least one embodiment of a method of treating a patient of the present disclosure, the method comprises steps of introducing a nucleotide segment encoding hepatocyte growth factor (HGF) into at least one adipose stromal cell (ASC), and administering the at least ASC to treat a patient in need. Optionally, the step of introducing a nucleotide segment may cause the level of HGF in the at least one adipose stromal cell to increase. The nucleotide segment of an embodiment of the present disclosure may be a viral vector. Additionally, the step of administering the at least ASC of the present disclosure to treat a patient in need may comprise intravenously injecting the at least one mammalian stem cell into the patient, where the patient may be a mammal, such as a human. Further, the step of administering the at least one ASC of the present disclosure causes the regeneration of at least one tissue of the patient, and/or a relative increase in blood perfusion of an ischemic tissue in the patient.

In at least one embodiment of a kit for enhancing the efficacy of ASC cells of the present disclosure, the kit comprises a first container, a composition contained within the first container; wherein the composition comprises HGF, a second container comprising a bolus of ASC, and instructions for treatment of the bolus of ASC with the composition.

### BRIEF DESCRIPTION OF THE DRAWINGS

The above-mentioned embodiments and other features, advantages and disclosures contained herein, and the manner of attaining them, will become apparent and the present disclosure will be better understood by reference to the following description of various exemplary embodiments of the present disclosure taken in conjunction with the accompanying drawings, wherein:

FIG. 1A shows a flowchart depicting the steps for a method of cell-based therapy according to at least one embodiment of the present disclosure;

FIG. 1B shows a flowchart depicting the steps for a method of treating a patient according to at least one embodiment of the present disclosure;

FIG. 1C shows a flowchart depicting the steps for a method of cell-based therapy according to at least one embodiment of the present disclosure;

FIG. 1D shows a flowchart depicting the steps for a method of cell-based therapy according to at least one embodiment of the present disclosure;

FIG. 2 shows graphical and visual representations of the reduction of hepatocyte growth factor (HGF) expression by adipose stromal cells (ASC) following transduction with short hairpin RNA (shRNA), according to at least one embodiment of the present disclosure;

FIG. 3 shows visual and graphical representations of relative levels of phosphorylated Akt (pAKT) to total Akt in ASC-shHGF and ASC-shCtrl cells in response to serum deprivation according to at least one embodiment of the present disclosure;

FIG. 4 shows the graphical representation of levels of green fluorescent protein (GFP)-labeled shCtrl- and shHGF-ASC in ischemic and non-ischemic muscles at 1, 5 and 20 days after cell infusion, according to at least one embodiment of the present disclosure;

FIG. 5 shows the graphical representation that suppression of HGF increases the frequency of ASC undergoing apoptosis in ischemic but not in normal tissues, according to at least one embodiment of the present disclosure;

FIG. 6 shows a Western blot detection of c-Met phosphorylation in ASC, according to at least one embodiment of the present disclosure;

FIG. 7 shows a graphical representation of the efficacy of ASC following HGF pretreatment, according to at least one embodiment of the present disclosure; and

FIG. 8 shows (A) a Western blot analysis and (B) a graphical representation of hydrogen peroxide induced phosphorylation of c-Met in human ASCs.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The disclosure of the present application provides various methods for the alteration of the protein activity of adipose stromal cells (“ASC”). The methods disclosed herein increase the efficacy of ASCs as they relate to therapeutic treatment of patients.

There exists an important need for advancement of cell-based therapies due to mammalian diseases and conditions which are currently untreatable or undertreated with current therapies. Given that the levels of cells required for effective cell-based therapies may not available, or secondary considerations may prohibit the use of large numbers of cells, a need exists for increasing the efficacy and safety of cell-based therapies.

While ASCs secrete many beneficial factors and promote reperfusion and tissue repair in ischemia models, knock-down of hepatocyte growth factor (HGF) by RNA interference (RNAi) attenuates ASC potency in vivo in a murine hindlimb ischemia model. Compared to ASC expressing normal levels of HGF, modified ASC demonstrate reduced persistence in repaired tissues. A series of in vitro and in vivo experiments described herein show that c-Met acts in promoting ASC-mediated repair of ischemic tissues and is part of a previously uncharacterized HGF/c-Met autocrine loop.

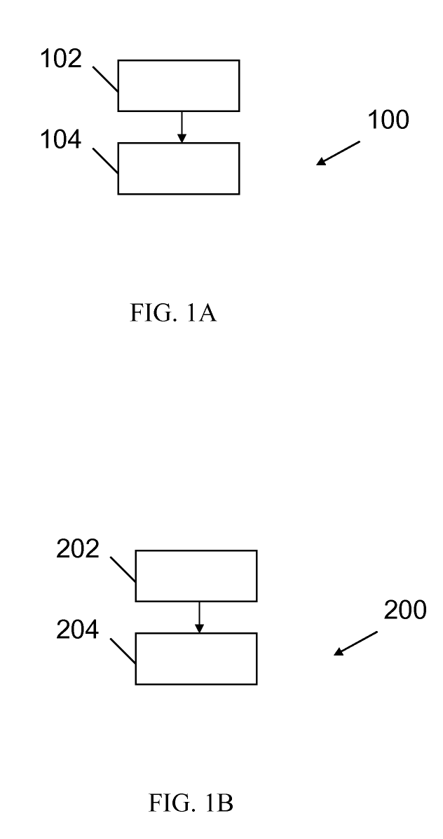

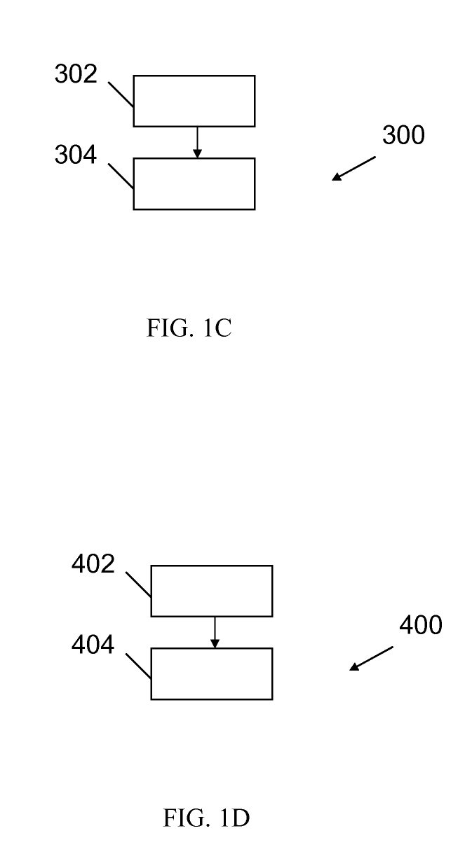

Turning to FIG. 1A, at least one embodiment of a method 100 of cell-based therapy is depicted. Exemplary method 100 comprises the steps of exposing at least one mammalian stem cell expressing mesenchymal-epithelial transition factor (c-Met) to a compound operable to phosphorylate c-Met (exemplary exposing step 102), and administering the at least one mammalian stem cell to a patient (exemplary administering step 104). Optionally, the compound may be a biological compound. The patient, in an exemplary embodiment, may have a vascular deficiency.

In an exemplary embodiment of the method 100 of cell therapy, the step 104 of administering the at least one mammalian stem cell treats the patient's vascular deficiency. Additionally, the at least one mammalian stem cell may be originally isolated from the patient. The patient may in at least one embodiment may be a non-human or human mammal. Further, the at least one mammalian stem cell may, in at least one embodiment, be isolated from a source selected from the group consisting of adipose tissue, peripheral blood, blood vessels, and umbilical cord blood. Additionally, the at least one mammalian stem cell, in at least one exemplary embodiment, comprises an adipose stromal cell. Further, according to at least one exemplary embodiment, the at least one mammalian stem cell may comprise a mesenchymal cell. The mesenchmal cell may be derived from any available cell type, including, but not limited to, bone marrow, skin, and myocytes.

In an exemplary embodiment of the method 100 of cell therapy, the compound may be hepatocyte growth factor (HGF), a fragment thereof which is operable to bind to c-Met, or a monoclonal antibody specific to c-Met.

In an exemplary embodiment of the method 100 for cell therapy, the step 102 of exposing the at least one mammalian cell may be for at least one minute, fifteen minutes, or one hour. Additionally, the step 104 of administering may comprise intravenously injecting the at least one mammalian stem cell into the patient. Further, the step 104 of administering may cause the regeneration of at least one tissue of the patient and/or the relative increase in blood perfusion of an ischemic tissue in the patient

According to a further exemplary embodiment of the method 100 for cell therapy, the compound may be a c-Met agonist. The c-Met agonist, in at least one embodiment, may be a small molecule or a nucleic acid. Additionally, in an exemplary embodiment, the compound may be a stressing agent. The stressing agent, in at least one embodiment, may comprise hydrogen peroxide.

Turning to FIG. 1B, at least one embodiment of a method 200 of treating a patient is depicted. Exemplary method 200 comprises the steps of exposing at least one adipose stromal cell to an effective amount of an agonist for mesenchymal-epithelial transition factor (c-Met) (exemplary exposing step 202), and administering the at least one adipose stromal cell to treat a patient in need (exemplary administering step 204). The at least one mammalian stem cell may be isolated from a source selected from the group consisting of adipose tissue, peripheral blood, blood vessels, and umbilical cord blood. Additionally, the at least one mammalian stem cell may in at least one embodiment be isolated from a non-human or human mammal, such as the patient.

In an exemplary embodiment of method 200, the at least one adipose stromal cell is capable of regenerating at least one tissue of the patient, and/or causing a relative increase in blood perfusion of an ischemic tissue in the patient.

Turning to FIG. 1C, at least one embodiment of a method 300 of cell-based therapy is depicted. Exemplary method 300 comprises the steps of exposing at least one mammalian stem cell expressing mesenchymal-epithelial transition factor (c-Met) to an environmental condition operable to phosphorylate c-Met (exemplary exposing step 302), and administering the at least one mammalian stem cell to a patient (exemplary administering step 304). In at least one exemplary embodiment of method 300, the environmental condition is selected from a group consisting of hypoxia, and serum starvation.

Turning to FIG. 1D, at least one embodiment of a method 400 of cell-based therapy is depicted. Exemplary method 400 comprises the steps of introducing a nucleotide segment encoding hepatocyte growth factor into at least one adipose stromal cell (exemplary introducing step 402), and administering the at least one adipose stromal to treat a patient in need (exemplary administering step 402). The nucleotide segment, according to an exemplary embodiment of method 400, causes the level of HGF in the at least one adipose stromal cell to increase. In an exemplary embodiment of method 400, the nucleotide segment may be a viral vector. Additionally, the at least one mammalian stem cell in at least one embodiment may be isolated from a non-human or human mammal, such as the patient.

In an exemplary embodiment of treating a patient according to an embodiment of the present disclosure, the at least one adipose stromal cell is capable of regenerating at least one tissue of the patient, and/or causing a relative increase in blood perfusion of an ischemic tissue in the patient. Further, the step of administering the at least one mammalian stem cell into the patient, according to at least one embodiment of the present disclosure, may be through intravenous injection.

According to at least one embodiment of a kit for enhancing the efficacy of ASCs in the treatment of a patient of the present disclosure, the kit comprising a first container, a composition contained within the first container, wherein the composition comprising HGF. In at least this exemplary embodiment, the kit further comprises a second container comprising a bolus of ASC, and instructions for treatment of the bolus of ASC with the composition.

### Methods

Dual-cassette lentiviral vectors, expressing green fluorescent protein (GFP) and either a small hairpin RNA (shRNA) specific for HGF mRNA (shHGF) or a control sequence (shCtrl), were used to stably transduce ASCs (ASC-shHGF or ASC-shCtrl). Activation of c-Met was blocked by incubating ASC for 1 hr in the presence of 10 p.M of the selective inhibitor PHA-665752 (Pfizer). Hyper-stimulation of c-Met phosphorylation was accomplished by treating for 1 hr with 100ng/ml purified recombinant HGF (R&D Systems). After both treatments, cells were washed with PBS before infusion. Cells were infused by tail vein infusion 24 hr after surgically inducing unilateral hindlimb ischemia in immunocompromised mice (N>6 /group). Reperfusion was monitored by laser Doppler perfusion imaging (Moor Systems). GFP cells were quantitated by fluorescent microscopic imaging of thin sections from the gastrocnemius muscles of ischemic and non-ischemic limbs. Apoptosis of GFP cells was measured in situ with an ApopTag kit (Chemicon). The relative levels of phosphorylated-Akt (p-Akt) and total Akt were determined by Western blotting with antibodies specific to each (Upstate).

### EXAMPLES

### Example 1

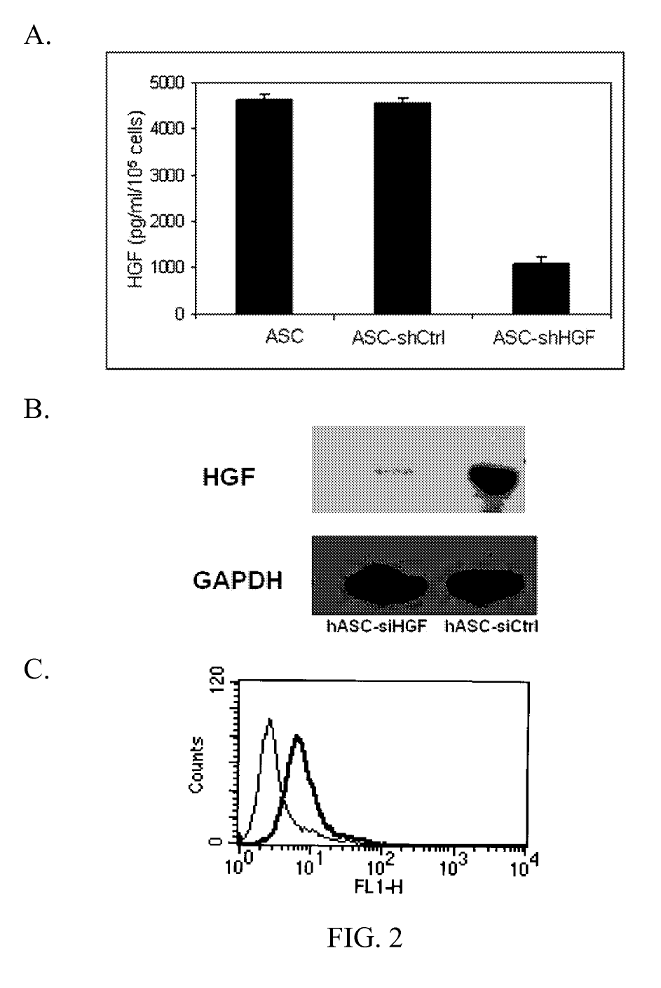

The levels of HGF expressed by ASCs are reduced by shRNA. ASC stably transduced with an shRNA targeted to the HGF gene secrete more than 4-fold less HGF into the medium during culture compared to ASC-shCtrl cells (FIG. 2A). Levels of HGF secreted into the medium were determined over a twenty-four period by ELISA. The effect of the shRNA was much larger when cell-associated HGF was assessed (FIG. 2B), as determined over an identical twenty-four hour period. Further, the receptor for HGF has also been shown to be expressed on the surface of ASCs by flow cytometry analysis (FIG. 2C).

### Example 2

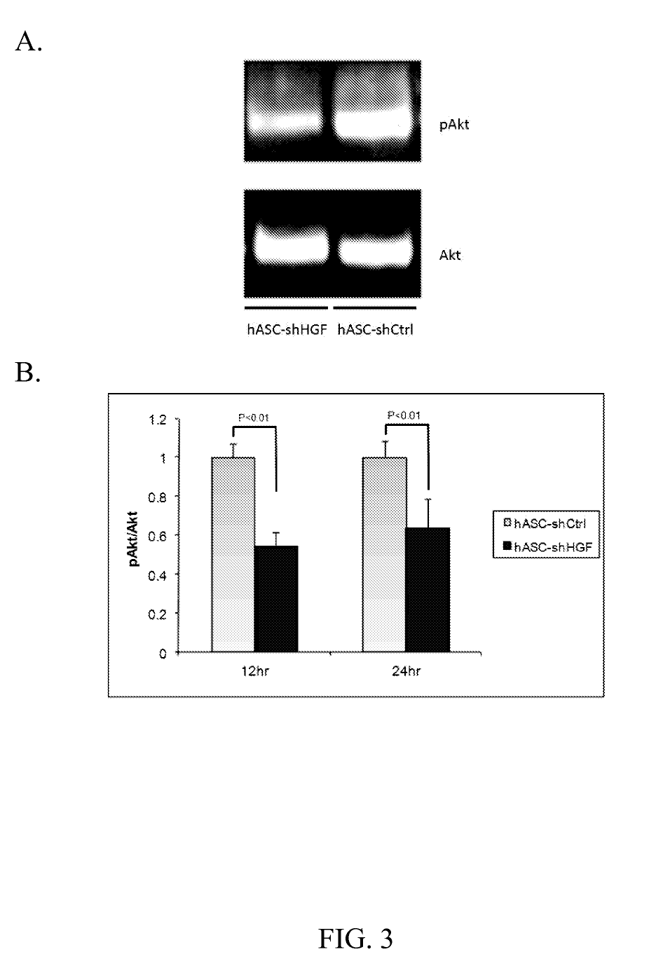

The pro-survival action of activated c-Met is transduced in part through PI3K/Akt, which plays a critical role in controlling survival and apoptosis. Western blot analysis of total and phosphorylated Akt (pAkt) after subjecting cells to the stress of serum deprivation for twelve hours indicated that the level of pAkt was reduced by 2-fold (p<0.01) in ASC-shHGF cells compared to ASC-shCtrl (FIG. 3). Panel B of FIG. 2 shows the densitometric quantitation of bands in panel A, as well bands from western blot analysis after twenty-four hours of serum deprivation.

### Example 3

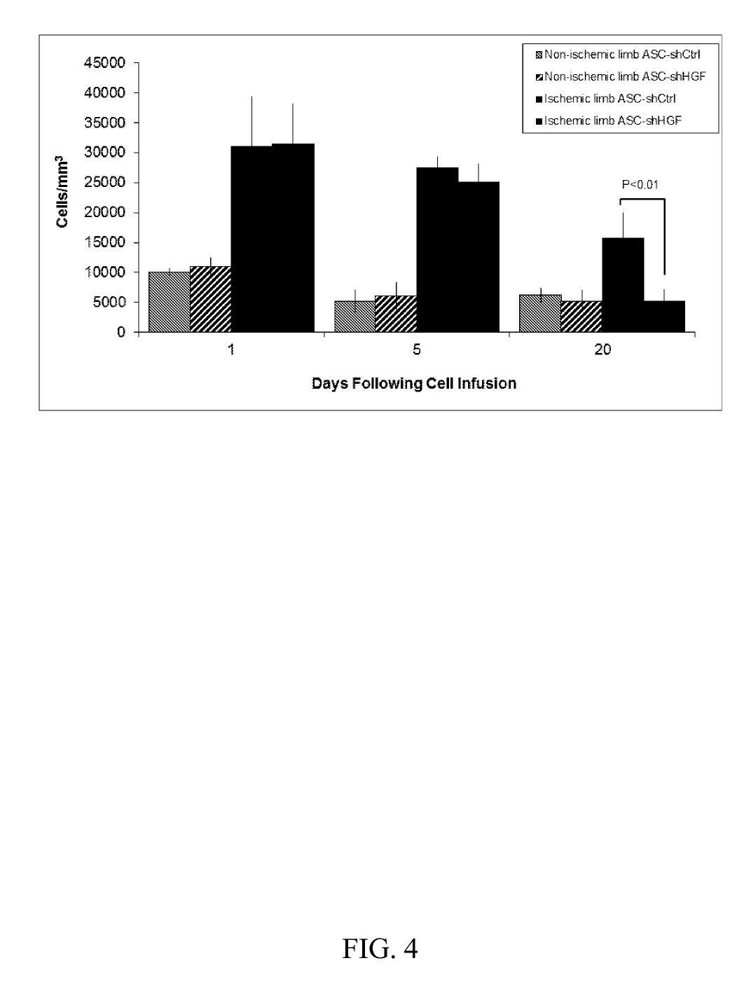

While reduced HGF expression in ASC does not appear to significantly affect the homing of ASCs to the injury site, the reduction in HGF does affect the persistence in injured tissues in vivo. The results of these in vivo experiments showed that there was no difference in total GFP-positive cells in ischemic limbs at 5 days after infusion of 10⁶ ASC, indicating similar homing potentials (FIG. 4).

Detection of GFP-labeled shCtrl- and shHGF-ASC in ischemic and non-ischemic muscles was conducted at 1, 5 and 20 days following cell infusion. At the indicated times after cell infusion 3 mice were sacrificed and the gastrocnemius muscles from both limbs were removed and fixed. The entire muscle was sectioned into 10-15 thin sections, each separated by 100-gm intervals. Thin sections (5 gm) were stained with GFP and anti-smooth muscle a-actin antibodies. Sections were also stained with 4′,6-diamidino-2-phenylindole dihydrochloride to visualize nuclei. Images of sections were obtained at 400× magnification. Ten randomly selected images were analyzed with Image J software.

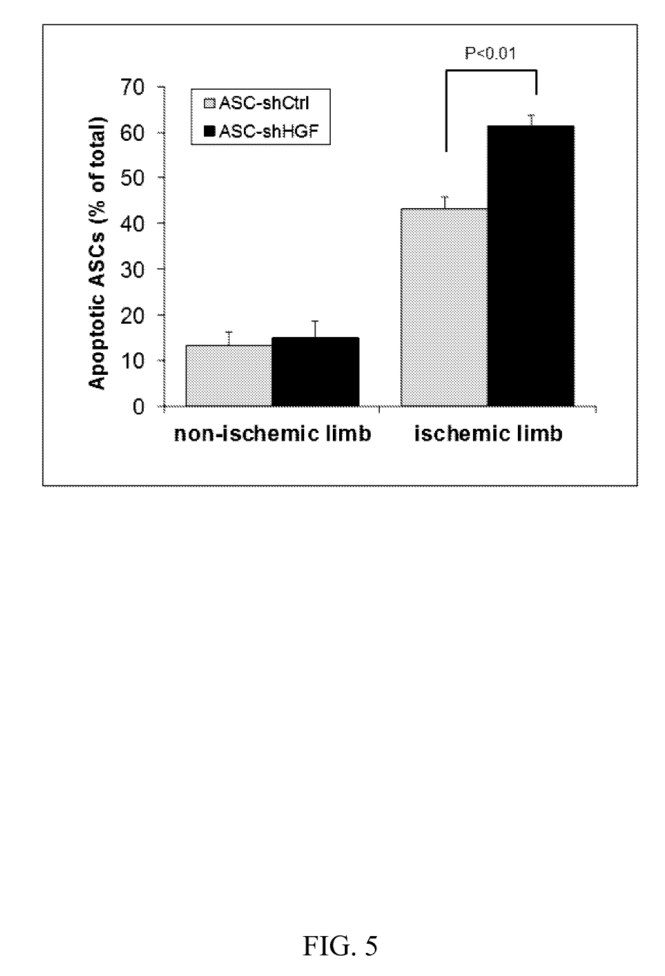

Suppression of HGF increases the frequency of ASC undergoing apoptosis in ischemic but not normal tissues. However, significantly more ASC-shHGF cells were apoptotic than ASC-shCtrl cells (61+0.1% vs. 41%+3.2%, respectively, P<0.01) (FIG. 5). Tunnel-positive as well as total ASC were quantitated in thin sections of ischemic and non-ischemic gastrocnemius muscles of 3 mice from each treatment group taken 5 days after infusion.

Further, there was no significant difference in apoptosis percentages in normal tissues (13.1±6.8% and 14.9±6.4% for ASC-shCtrl and ASC-shHGF, respectively). By 20 days following infusion, 3-fold fewer ASC-shHGF were present in ischemic tissues compared to ASC-shCtrl (p<0.01) (FIG. 4).

### Example 4

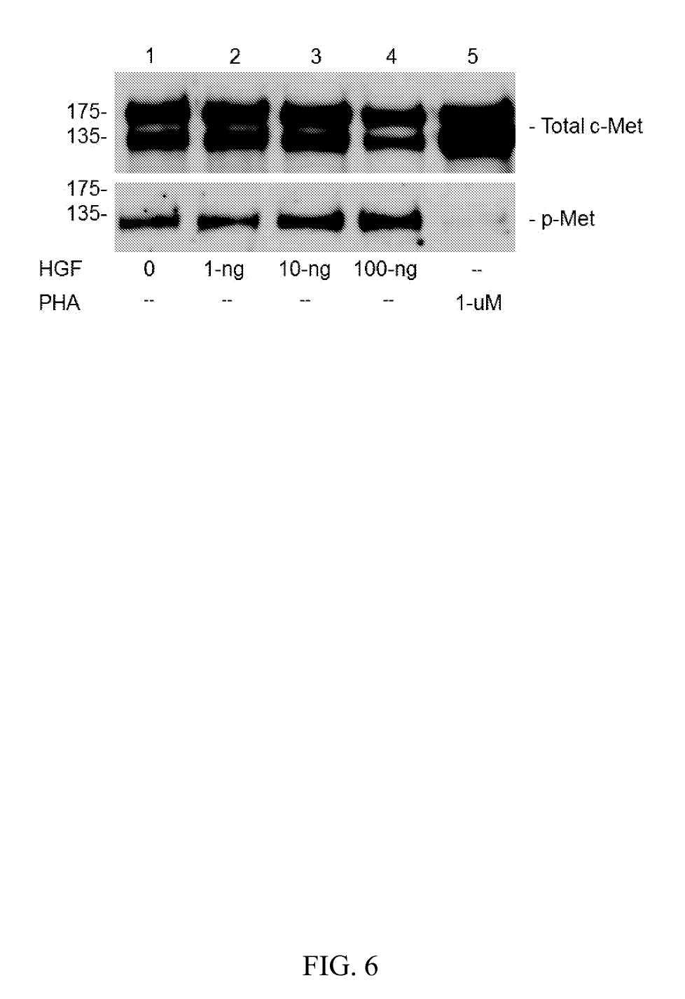

ASCs possess a functional HGF/c-Met autocrine loop that is influential in cellular survival in adverse environments. Disruption of this cycle has reduced the ability of ASC to withstand stresses associated with the ischemic environment and, thus, their ability to affect tissue rescue and repair. The link between the HGF/c-Met autocrine loop and survival under adverse conditions was examined directly either by specifically inhibiting c-Met phosphorylation with the selective inhibitor PHA-665752 or, conversely, by stimulating greater phosphorylation with excess HGF (FIG. 6). FIG. 5 shows the detection of c-Met phosphorylation in ASC by Western blot analysis. Upper panel: total c-Met protein. Lower panel: phosphorylated c-Met (p-Met). A basal level of p-Met phosphorylation is observed in cultured ASC (lane 1). Phosphorylation is stimulated in a dose dependent manner by incubating the cells for 15 minutes with HGF (lanes 2-4). Basal p-Met levels are reduced by treating the cells for 15 minutes with the inhibitor PHA-665752 (PHA) (lane 5).

### Example 5

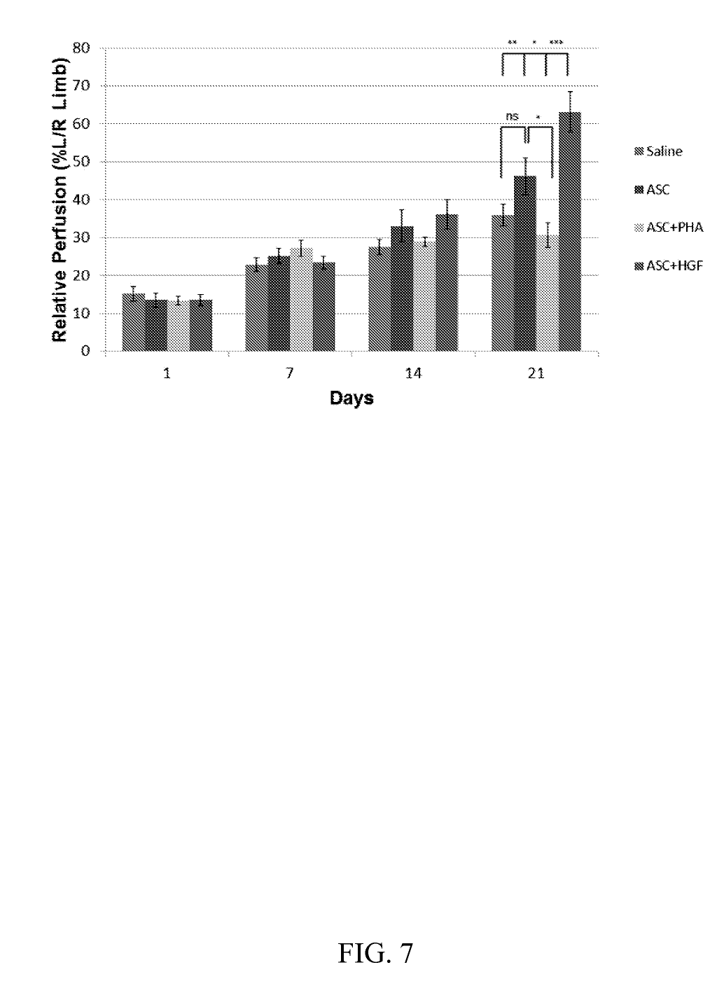

The efficacy of ASC can be enhanced by HGF pretreatment. Hindlimb ischemia was surgically induced in immunotolerant NS2 mice (N=6/group). At 24 hours after surgery the mice received tail vein injections of saline control or 3×10⁵ untreated ASC or the same number of ASC that had been treated for 1 hour with PHA or HGF (FIG. 7). The relative blood perfusion of ischemic (left) and non-ischemic (right) limbs was determined by laser Doppler perfusion imaging on the days indicated. *, P<0.05; **, P<0.01, ***, P<0.001. ns, not significant. At 21 days the relative (ischemic to non-ischemic) hindlimb perfusion of PBS-treated control mice was 35.9+2.9%. The low dose of untreated ASC induced only 46.8±4.3% relative perfusion, which was greater than control, but not significant (P>0.05). Pretreating ASC with HGF enhanced the potency of the non-efficacious dose, leading to significant reperfusion (63.2+5.3%; P<0.001 vs PBS and P<0.05 vs untreated ASC). Conversely, inhibiting c-Met abolished the effect of ASC on hindlimb reperfusion (30.3+2.1%).

In addition to the paracrine effects of HGF expression on recovery of host tissues from ischemic insult described above, these results establish that HGF is necessary for autocrine promotion of ASC survival and consequent efficacy. Importantly, we have shown in at least one embodiment that increased survival and potency can be attained with only a brief and reversible treatment immediately before administration.

### Example 6

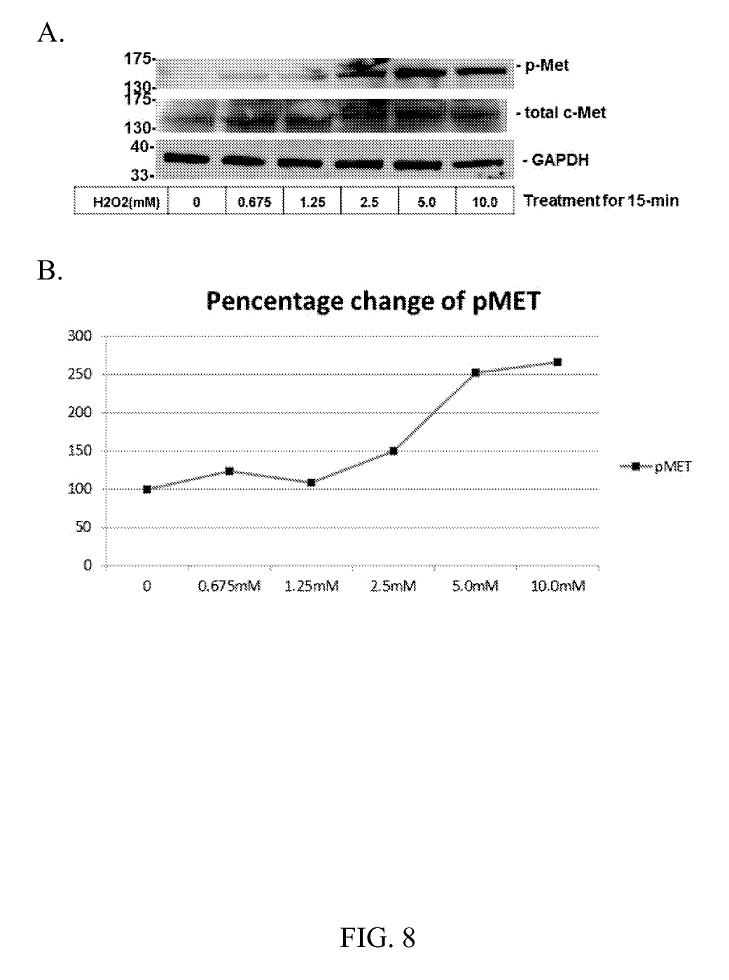

Treatment of human ASC (h-ASC) under various environmental conditions may increase the level of phosphorylation of c-Met. Exposure of h-ASC to hydrogen peroxide is shown herein to increase the level of phosphorylation of c-Met (See FIGS. 8A and B). Samples of h-ASC were exposed to levels of hydrogen peroxide from 0 to 10mM. These levels of exposure included 0, 0.675, 1.25, 2.5, 5.0, and 10.0 mM of hydrogen peroxide. Western blot analysis of treated cells is shown in FIG. 7A. The Western blot analysis utilized probes for phosphorylated c-Met (p-Met), as well as c-Met, and glyceraldehyde 3-phosphate dehydrogenase (GAPDH). FIG. 8B shows the percentage change of p-Met due to exposure to varying levels of hydrogen peroxide as compared to untreated h-ASC.

The methods of modification of ASC of the present disclosure have various benefits to the treatment of patient diseases and conditions. For example, the phosphorylation of c-Met by HGF may increase the efficacy of ASC when used as a therapeutic in a patient.

While various embodiments of methods for the treatment of ASCs, and patients, have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the spirit and scope of the present disclosure.

### REFERENCES

Cal L, Johnstone B H, Cook TG, Liang Z, Traktuev D, Cornetta K, Ingram D A, Rosen E D, March K L, Suppression of hepatocyte growth factor production impairs the ability of adipose stromal cells to promote ischemic tissue revascularization., Stem Cells, 2007, 25(12)3234-43.

We claim:

1. A method of cell-based therapy, the method comprising the steps of:
exposing at least one mammalian stem cell expressing mesenchymal-epithelial transition factor (c-Met) to a compound operable to phosphorylate c-Met; and
administering the at least one mammalian stem cell to a patient.

2. The method of claim 1, wherein the patient has a vascular deficiency.

3. The method of claim 2, wherein the step of administering the at least one mammalian stem cell treats the patient's vascular deficiency.

4. The method of claim 1, wherein the at least one mammalian stem cell is originally isolated from the patient.

5. The method of claim 1, wherein the at least one mammalian stem cell is isolated from a source selected from the group consisting of adipose tissue, peripheral blood, blood vessels, and umbilical cord blood.

6. The method of claim 1, wherein the at least one mammalian stem cell comprises an adipose stromal cell.

7. The method of claim 1, wherein the compound is hepatocyte growth factor (HGF).

8. The method of claim 1, wherein the compound is a fragment of HGF operable to bind to c-Met.

9. The method of claim 1, wherein the compound is a monoclonal antibody specific for c-Met.

10. The method of claim 1, wherein the compound is a c-Met agonist.

11. The method of claim 10, wherein the c-Met agonist is a small molecule.

12. The method of claim 10, wherein the c-Met agonist is a nucleic acid.

13. The method of claim 1, wherein the compound is a stressing agent.

14. The method of claim 13, wherein the stressing agent comprises hydrogen peroxide.

15. The method of claim 1, wherein the step of exposing the at least one mammalian cell is for at least one minute.

16. The method of claim 1, wherein the step of exposing the at least one mammalian cell is for at least fifteen minutes.

17. The method of claim 1, wherein the step of exposing the at least one mammalian cell is for at least about one hour.

18. The method of claim 1, wherein the step of administering the at least one mammalian stem cell to a patient comprises intravenously injecting the at least one mammalian stem cell into the patient.

19. The method of claim 1, wherein the patient is a mammal.

20. The method of claim 1, wherein the patient is a human.

21. The method of claim 1, wherein the step of administering the at least one mammalian stem cell to a patient causes the regeneration of at least one tissue of the patient.

22. The method of claim 1, wherein the step of administering the at least one mammalian cell causes a relative increase in blood perfusion of an ischemic tissue in the patient.

23. A method of cell-based therapy, the method comprising the steps of:
exposing at least one mammalian stem cell expressing mesenchymal-epithelial transition factor (c-Met) to an environmental condition operable to phosphorylate c-Met; and
administering the at least one mammalian stem cell to a patient.

24. The method of claim 23, wherein the environmental condition is selected from a group consisting of hypoxia, and serum starvation.

25. A method of treating a patient, the method comprising the steps of:
exposing at least one adipose stromal cell to an effective amount of an agonist for mesenchymal-epithelial transition factor (c-Met); and
administering the at least one adipose stromal to treat a patient in need.

26. The method of claim 25, wherein the at least one mammalian stem cell is isolated from a source selected from the group consisting of adipose tissue, peripheral blood, blood vessels, and umbilical cord blood.

27. The method of claim 25, wherein the at least one mammalian stem cell is isolated from the patient.

28. The method of claim 25, wherein the at least one adipose stromal cell is capable of regenerating at least one tissue of the patient.

29. The method of claim 25, wherein the step of administering the at least one mammalian cell causes a relative increase in blood perfusion of an ischemic tissue in the patient.

30. A method of treating a patient, the method comprising the steps of:
introducing a nucleotide segment encoding hepatocyte growth factor (HGF) into at least one adipose stromal cell (ASC); and
administering the at least one ASC to treat a patient in need.

31. The method of claim 30, wherein the step of introducing a nucleotide segment causes the level of HGF in the at least one ASC to increase.

32. The method of claim 31, wherein the nucleotide segment is a viral vector.

33. The method of claim 30, wherein the step of administering comprises intravenously injecting the at least one mammalian stem cell into the patient.

34. The method of claim 30, wherein the patient is a mammal.

35. The method of claim 30, wherein the patient is a human.

36. The method of claim 30, wherein the step of administering the at least one ASC causes the regeneration of at least one tissue of the patient.

37. The method of claim 30, wherein the step of administering the at least one ASC causes a relative increase in blood perfusion of an ischemic tissue in the patient.

38. A kit for enhancing the efficacy of ASC cells, the kit comprising:
a first container;
a composition contained within the first container, wherein the composition comprises HGF;
a second container comprising a bolus of ASC; and
instructions for treatment of the bolus of ASC with the composition.
